(19) United States
(12) Patent Application Publication
(10) Pub. No.: US 2024/0231997 A1
(43) Pub. Date: Jul. 11, 2024

(54) METHODS AND SYSTEMS FOR SECURE AND RELIABLE INTEGRATION OF HEALTHCARE PRACTICE OPERATIONS, MANAGEMENT, ADMINISTRATIVE AND FINANCIAL SOFTWARE SYSTEMS

(71) Applicant: ARTHUR INTELLIGENCE INC., MONTREAL (CA)

(72) Inventors: Fredrik HAARD, STOCKHOLM (SE); Philippe HEBERT, MONTREAL (CA); SIVAN ALTINAKAR, MONTREAL (CA)

(21) Appl. No.: 18/261,752

(22) PCT Filed: Jan. 18, 2021
(86) PCT No.: PCT/CA2022/050066

§ 371 Date: Jul. 17, 2023

Related U.S. Application Data
(60) Provisional application No. 63/199,686, filed on Jan. 18, 2021.

(51) Int. Cl. G06F 11/07; G06F 11/30

(52) U.S. Cl. CPC G06F 11/0793; G06F 11/3048

(57) ABSTRACT

Known extraction or mutation of information enclosed in data sources associated with workflow software in a reliable, resilient and secure fashion at scale is a complex task. According to the present invention, there are provided methods and systems for improving reliability and resiliency of a computer-based software system installed on an object infrastructure managed by an external party. One method includes: packaging a computer-based system for as an executable asset; installing an executable asset of a first parent module as an operating system service; installing an executable asset of a second parent module as an operating system primitive; configuring the first parent module to execute said computer-based system as a child process; configuring the first and second parent process to respectively execute a child process for monitoring health of the other parent module and child processes, and attempting recovery of detected failures.

### FIELD OF THE INVENTION

The present disclosure relates to systems and methods to integrate software systems dedicated to the operations, management, administration, and/or financial management of healthcare practices as well as software systems dedicated to the management of electronic health records or electronic medical records.

### Definitions

Tenant: A tenant is a group of users who share a common access with specific privileges to a software system and its component, including data, configurations, infrastructure resources, etc. A tenant generally maps to an entity such as a company or a public sector organization engaging in business with a provider of said software system to use said software system. In this disclosure example tenants may include private or public medical practice, medical facilities, etc.

Data source: Any first, second or third-party software system dedicated to either (i) the operations, management, administration, and/or financial management of healthcare practices, (ii) the management of patient health insurance and processing health insurance claims or (iii) the management of electronic health records or electronic medical records.

Data extractor: A system configured to extract data enclosed within one or more data sources to one or more destination, or mutate data enclosed within said one or more data sources.

Mutation, mutating: Mutation refers to the C, U, D in CRUD—Create, Update, Delete (data).

Infrastructure resource: An infrastructure resource refers to a computer infrastructure resource, such as a data storage, servers, databases, networking, software service, content delivery networks, etc., wherein said resource may be hosted on virtualized or physical machines. It is a generalization of the concept of “cloud resource”, to include computer infrastructure resources on the edge.

Subject infrastructure: Following the definition of subject and object in English grammar, where a subject is the entity performing an action, and the object is having the action performed upon/on/using it, subject infrastructure defines a computer system infrastructure composed of various embodiments of infrastructure resources, said subject infrastructure performing action on an object (in this disclosure at least one object infrastructure). Said subject infrastructure can be in the cloud or on the edge.

Object infrastructure: Following the definition of subject and object in English grammar, where a subject is the entity performing an action, and the object is having the action performed upon/on/using it, object infrastructure defines a computer system infrastructure composed of various embodiments of infrastructure resources, said object infrastructure having an action performed on it by a subject infrastructure. Said object infrastructure can be in the cloud or on the edge.

Isolation (logical, physical): Physical isolation is defined as the physical isolation of a component (device, data, etc.) via lack of connectivity to a network. Logical isolation is defined as presence of isolation via a protocol, device or some sort of software protection.

Long-lived connection: A long-lived connection is a connection that is kept open for much longer than required for a single transaction or operation. A long-lived connection can be kept for minutes, hours, days or longer, as necessary.

Full-duplex connection: A full-duplex connection is a connection that enables both parties engaging in the connection to send message to the other party, whether simultaneously or non-simultaneously.

Real-time: A real-time operation is an operation that is required to be performed within a specified deadline. Deadlines may vary from system to system. In this disclosure, real-time implies a deadline of less than 5 seconds.

Near real-time: A near real-time operation is an operation that are required to be performed within time constraints defined as an interval. Said range is generally more permissive than real-time deadlines. In this disclosure, near real-time implies a time constraint interval of between 5 seconds and 30 minutes.

Storage: In this disclosure, storage refers to persistent or volatile storage, examples of such storage being file system, object storage systems (such as Google Cloud Storage, Amazon Web Services S3, Azure Cloud Storage, etc.), key-value databases, wide-column databases, document databases, relational databases, etc.

Storage object: Generalization of a storage primitive; named after the unit of storage of object storage systems such as Google Cloud Storage, Amazon Web Services S3, and Azure Cloud Storage, but refers to any such storage primitive that can store data—file, table row, document, key+value entry, etc.

Executable asset: An executable asset is any file or object that can be executed by the operating system on which it resides. Examples of executable assets include executable binaries (e.g. .exe files), executable scripts (e.g. .sh, .batch files), etc.

### BACKGROUND OF THE INVENTION

Healthcare practices usually use one or many workflow software to manage their operations, whether medical, financial, administrative, human, inventory, insurance, analytical or otherwise, as well as to manage the electronic records of their patients. As defined herein, a practice is a business through which one or more physicians practice medicine.

Said workflow software are typically associated with a data store or data registry, together named “data source” in this disclosure. Said data sources generally include a wealth of information that can be extracted and processed to offer a variety of valuable services to the medical and non-medical staff of the practice. Moreover, there is high commercial value in being able to mutate information enclosed in said data sources to provide complementary services to patients or users of said workflow software.

The extraction or mutation of the information enclosed in the data sources associated with said workflow software in a reliable, resilient and secure fashion at scale is a complex task. Significant engineering research and development is required for a party that is external to the practices to be able to integrate these data sources across a large number of practices.

This significant engineering research and development is due to four major sources of engineering complexity.

The first major source of engineering complexity is the heterogeneity of the infrastructure on which said workflow software are operated. Indeed, most healthcare practices operate their workflow software on their own computer system hardware, located on the physical premises of their practice. This in turn increases the complexity of the integration by an external party whose infrastructure is external to that of the practice, as they do not have control or reliable physical or virtual access to the computer infrastructure of the practice. Often these on-premise infrastructures are operated by different IT firms from practice to practice and standardization of the software or hardware environment is not feasible. The high variability of these infrastructure configurations and security measures creates a wide range of combinations which can result in failure of the external party to offer a reliable, resilient and secure offering.

The second major source of engineering complexity comes from the heterogeneity of the workflow software across the healthcare practices. Indeed, in order to reach as wide of a market as possible, an external party must integrate a variety of competing and/or complementary workflow software. The data sources of said workflow software may use technologies with interfaces, protocols or drivers that are incompatible with each other. Said data sources range from outdated legacy systems that are no longer supported by its manufacturer to state-of-the-art systems. Furthermore, some data sources may only support a subset of the functionalities required for the extraction or mutation of the information enclosed within, or the parties managing said workflow software may only grant limited permissions or accessibility to said data sources.

The third significant source of engineering complexity is security compliance and tenancy isolation. Indeed, due to the sensitive nature of the data extracted, an external party must provide strong guarantees of security and isolation of the data. In the case of healthcare data, this is even more important as protected health information (PHI) may be part of the data extracted. As such, an ideal extraction or mutation system must observe high levels of compliance to regulatory norms or laws such as The Health Insurance Portability and Accountability Act of 1996 (HIPAA), effective in the United States, and The Personal Information Protection and Electronic Documents Act (PIPEDA), effective in Canada.

The fourth significant source of engineering complexity comes from the necessity of designing a system to facilitate the deployment of new functionalities, updates and corrections in an autonomous way with confidence, without the need for a representative of the external party to interact with the external infrastructure or any software installed therein.

Known prior art discloses tools and methods that partially responds to the needs above, but our research has not been able to demonstrate the existence of prior art that responds to all the criteria elicited above.

Our research compiled prior art in five major classes of software systems which come closest to the use-case and requirements elicited above:
1. Visual Analytic Platforms
2. Internet of Things (IoT) Platforms
3. Database Replication tools
4. Data Integration platform and tools
5. Healthcare Integration Engines/Healthcare Interface Engines

### 1. Visual Analytic Platforms (VAP)

Visual Analytic Platforms are platforms dedicated to providing powerful data visualization tools against various data sources.

VAPs tackle the workflow software heterogeneity issue by offering out of the box integrations with various data sources as part of their offering. While most of them offer a generous number of integrations, they are not guaranteed to have support for all workflow systems, especially legacy ones.

Moreover, VAPs present shortcomings that make them unfit for the use case of this disclosure:
a. VAPs are dedicated to the curation of data through visualizations and analytics and are not built to provide the ability to extract data from data sources and pipe it to a destination other than its own systems (whether software, hardware or infrastructural);
b. VAPs are not designed to support mutation of data within the data sources they are connected to;
c. None of the VAPs explored in our research showed elements of autonomy, reliability and resilience explored above as most of them integrate data sources either through a direct network connection or via the deployment of a dedicated server within the third party network, neither of which are possible based on the IT situation explained above.

Due to these shortcomings, we have evaluated VAPs not to support the use-case of this disclosure.

### 2. Internet of Things (IoT) Platforms

Internet of Things (IoT) Platform offerings support the integration of a wide, heterogeneous set of hardware and software infrastructure as their core value propositions.

This being said, IoT Platform offerings also assume that you have control over the hardware on which it is installed and that if your device fails, you have a way to have access to the device and repair it yourself or swap it with another.

IoT Platform researched, such as Microsoft Azure IoT, Google Cloud Platform Cloud IoT Core, and AWS IoT SDK xAWS Greengrass all make such an assumption, and thus come either as embeddable software development toolkit to be embedded in your own deployed code, or as managed solutions that manage the whole code lifecycle for the device. Neither of these solutions are adequate for the reality expressed above as neither tackle the reliability nor resiliency demands expressed above and assume access to the device is possible. Moreover, these platforms are generally not meant for expanding sets of features but instead are meant to establish a control-command pattern and a lightweight communication channel for devices with low-bandwidth requirements.

### 3. Database Replication Tools

Database replication tools are interesting solutions in that they address the first and second requirements quite well; they are generally built with reliability in mind since they serve the purpose of replicating or mirroring a database in near-real time to real time. Most are also designed to be installable on a variety of computer systems, which partially answers the problem of infrastructure heterogeneity.

This being said, database replication tools present shortcomings that make them unfit for the use case of this disclosure:
i. Database replication tools generally support only limited types of databases for replication. None of the database replication tools found in our research support the use case of integrating a custom REST, SOAP, or GraphQL API exposed by a workflow system provider; due to the legacy nature of some of the systems to integrate, it is not even guaranteed that the database replication tool can support all SQL-based workflow systems to integrate;
ii. Database replication tools may require elevated permissions against the source system in order to implement triggers against replicated tables, or in order to add metadata fields to better keep track of changes. Those permissions will most likely never be given out by the IT staff managing the workflow system or by the company producing it;
iii. Database replication tools do not support querying the local environment (operating system, network, etc.) for diagnostics and recovery, and are generally opaque to the target system in terms of performance or issues;
iv. Not all database replication tools support two-way mirroring. Hence database replication tools that do not support two-way mirroring do not support mutation of the data enclosed within the data source by the external infrastructure;
v. Database replication tools are not extendable by the first party system to add features or patch issues;
vi. Using database replication tools at scale may prove to be extremely expensive as the model of database replication tools require a 1-1 mirroring between a local database and a remote database, thereby requiring the external infrastructure to have one database instance for each of the healthcare practice software instance to integrate.

Due to these shortcomings, we have evaluated Database replication tools not to support the use-case of this disclosure.

### 4. Data Integration Platforms and Tools

The value proposition of Data Integration tools is simple: Integrate data sources seamlessly into destinations using managed pipelines. While this is an amazing value proposition, all the candidates we have researched focus mostly on integrating well known end-user, web-facing products, such as HubSpot, Google Sheets, GitHub, Google Analytics, etc. Our research also found support for many SQL and No-SQL databases, but all of these databases have to be hosted in a managed fashion on a known cloud provider platform. Examples of such databases are Amazon Aurora, Amazon RDS, GCP Cloud SQL, etc.

As such, none of the solutions found in this class seem to answer our primary need, which is to integrate disparate data sources dispersed across a wide range of (often self-hosted) heterogeneous infrastructure in a reliable fashion.

### 5. Healthcare Integration Engines/Healthcare Integration Engines (HIE)

Healthcare Interface Engines (HIE) solves the problem of sharing and exchanging data between healthcare systems by providing on-premise extraction-load-transform (ELT) tools that ingest data structured in the proprietary data schema of the workflow system data source, and output a standard-compliant data schema.

Healthcare Interface Engines are effectively on-premise mini ELT/ETL tools that expose APIs on the local network or the public internet to be interfaced with.

HIE interface with a wide range of data sources, and are designed to be secure, resilient and reliable on any infrastructure they support. From what our research has gathered, the results seem to show that the HIE may also be capable of self-diagnosis and diagnosis of their environment.

The HIE solutions, however, have the following drawbacks:
a. HIE are dedicated to the creation of an interoperability layer from the source schema to a target schema implementing one of the Healthcare Standards (HL7 V2, HL7 V3, FHIR, C-CDA, etc.) but is not dedicated with the extraction of the raw data format to a first party infrastructure for ingestion and processing;
b. HIE are not able to separate tenants within a same workflow system;
c. HIE do not support the extension of their feature set for custom purposes with the deployment of custom patches by the first party.

Based on (a), (b) and (c), we have evaluated that HIE are not adequate to answer particular use-cases.

There is therefore a need in the industry for covering the requirements expressed above.

### SUMMARY OF THE INVENTION

According to the present invention, there is provided method for improving reliability and resiliency of a computer-based software system installed on an object infrastructure managed by an external party, the method comprising:
a. packaging said computer-based software system as a first executable asset;
b. installing an executable asset of a first parent module as an operating system service on the object infrastructure, said first parent module being configured to operate at least one child module, each of said child module comprising a child process executing an executable asset selected from a first set of executable assets;
c. installing an executable asset of a second parent module as an operating system primitive on the object infrastructure, said second parent module being configured to operate at least one child module, each of said child module comprising a child process executing an executable asset from a second set of executable assets;
d. configuring the first parent module to include a child module executing the executable asset of the computer-based software system, and a child module executing a second executable asset being configured to monitor health of the second parent module and at least one child module thereof, and attempt recovery of detected failure;
e. configuring the second parent module to include one child module executing a third executable asset being configured to monitor health of the first parent module and at least one child module thereof, and attempt recovery of detected failure;
f. monitoring the health of the first parent module, the second parent module, and their respective at least one child module; and
g. attempting recovery of the detected failure in the first parent module, the second parent module, and their respective at least one child module.

In embodiments, the method further comprises:
h. configuring the first parent module to automatically update the executable assets of its at least one child module whenever a new executable asset for said at least one child module is published to a configured location, said update comprising executing a check suite to assert the new executable asset does not introduce a failure;
i. configuring the first parent module to automatically update the executable asset of its at least one child module whenever a new executable asset for said at least one child module is published to a configured location, said update comprising executing a check suite to assert the new executable asset does not introduce a failure;
j. automatically updating the executable asset of the at least one child module of the first parent module whenever a new executable asset for said at least one child module is published to a configured location, said updating comprising executing a check suite to assert the new executable asset does not introduce one or more failures; and
k. automatically updating the executable asset of the at least one child module of the second parent module whenever a new executable asset for said at least one child module is published to a configured location, said updating comprising executing a check suite to assert the new executable asset does not introduce one or more failures.

In embodiments, the method comprises:
h. notifying a subject infrastructure of a detected failure; and
i. notifying a subject infrastructure of a result of the attempted recovery in step (g).

According to the present invention, there is also provided a method for securely extracting or mutating data associated to a tenant in at least one data source located in an object infrastructure, from a subject infrastructure, the method comprising:
a. configuring a subject infrastructure provision logically isolated infrastructure resources dedicated to the tenant, said resources comprising a communication channel, a set of tenant data extraction IAM primitives, and a tenant logically isolated storage;
b. granting external access to said infrastructure resources to entities authenticating as a user comprised in said set of tenant data extraction IAM primitives;
c. writing an authentication credential for said user to a configuration distribution module, said configuration distribution module returning a single-use, high-entropy, unique key, “one time key”;
d. installing a computer-based software system, “data extractor” on the object infrastructure to perform extraction or mutation of said data associated to the tenant in the at least one data source;
e. configuring said data extractor to connect to said at least one data source;
f. configuring said data extractor to retrieve said authentication credential from said configuration distribution module, thereby using the single-use one time key, and providing said computer-based software system with access to the logically isolated infrastructure resources;
g. using said communication channel to communicate between the data extractor and the subject infrastructure to (i) receive extraction or mutation commands, (ii) execute said extraction or mutation commands, and (iii) respond where applicable; and
h. using said tenant logically isolated storage to upload extracted data to the subject infrastructure.

In embodiments, the method further comprises a recurrent and automatic rotation of said authentication credential, said rotation comprising:
i. distributing a new authentication credential to the tenant logically isolated storage;
j. communicating to the data extractor that a new authentication credential is available in the tenant logically isolated storage;
k. the data extractor downloading the new authentication credential to the object infrastructure;
l. the data extractor performing a check suite to assert that the new authentication credential has sufficient access privilege on the subject infrastructure to allow the data extractor to perform all of at least one function of said data extractor;
m. the data extractor communicating to the subject infrastructure that it has rotated its authentication credential with the new authentication credential; and
n. the subject infrastructure expiring the authentication credential rotated out by the data extractor.

According to the present invention, there is also provided a computer-based software system for extracting or mutating data in at least one data source associated to at least one tenant, said at least one data source being located on an object infrastructure, the system comprising:
a. at least one data extractor connectable to the at least one data source for extracting the data from said data source or mutating said data in the said data source, said at least one data extractor being installed on the object infrastructure;
b. a subject infrastructure connectable to the at least one data extractor, wherein the at least one data extractor communicates (i) data extracted from the at least one data source and (ii) a log of operations of the at least one data extractor to the subject infrastructure;
   wherein the at least one data extractor comprises:
a main module for performing the extraction or mutation of the data in the at least one data source, said main module comprising:
   a) a parent module executed as an operating system service process from a corresponding executable asset;
   b) a configuration file to store a configuration of the parent process;
   c) a plurality of child modules, each of which being separated from the parent module of the main module and from each other by each being executed from a corresponding executable asset as a child process of the process of the parent module of the main module;
a watchdog module for monitoring health of the main module and attempting recovery of detected failures in said main module, said watchdog module comprising:
   a) a parent module executed as an operating system primitive including an operating system service process or an operating system scheduled task process from a corresponding executable asset;
   b) a configuration file to store a configuration of the parent process;
   c) a plurality of child modules, each of which being separated from the parent module of the watchdog module and from each other by each being executed from a corresponding executable asset as a child process of the process of the parent module of the watchdog module;
the parent process of the main module, comprising:
   a) a heartbeat component for sending a heartbeat signal to the subject infrastructure to inform said subject infrastructure that the parent process of the main module of the at least one data extractor has liveness;
   b) a configuration and update component for
      i. updating the configuration files of the parent module of the main module, the executable assets of the plurality of child modules of the main module;
      ii. uploading the configuration file of the parent module of the main module to the subject infrastructure;
   c) a module orchestrator component for bootstrapping, starting, stopping and restarting the child processes of the plurality of child modules of the main module;
   d) a logging component for uploading logs of the parent module of the main module, and logs of the plurality of child modules of the parent module of the main module to the subject infrastructure;
the plurality of child modules of the main module, comprising:
   a) a watchdog module health monitoring and recovery module for recurrently performing a series of health check on the watchdog module, and attempting recovery of detected failures in the watchdog module;
   b) at least one data source integration module for extracting data from the at least one data source or mutating data within said data source;
the parent process of the watchdog module comprising:
   a) a heartbeat component for sending a heartbeat signal to the subject infrastructure to inform said subject infrastructure that the parent process of the watchdog module of the at least one data extractor has liveness;
   b) a configuration and update component for:
      i. updating the configuration file of the parent module of the watchdog module, the executable assets of the plurality of child modules of the watchdog module;
      ii. uploading the configuration file of the parent module of the watchdog module to the subject infrastructure;
   c) a module orchestrator component for bootstrapping, starting, stopping and restarting the child processes of the plurality of child modules of the watchdog module;
   d) a logging component for uploading logs produced by the parent module of the watchdog module, logs of the plurality of child modules of the parent module of the watchdog module to the subject infrastructure;
the plurality of child modules of the watchdog module, comprising:
   a) a main module health monitoring and recovery module for recurrently performing a series of health checks on the main module, and attempting recovery of detected failures in the main module:
      wherein the subject infrastructure comprises:
a storage comprising:
   a) the executable assets used by the at least one data extractor, said executable assets comprising the executable assets for the main module, watchdog module, and their respective components and child modules;
   b) a set of objects for each one of the at least one data extractor, each set of objects comprising:
      i. the configuration files of the at least one data extractor;
at least one communication channel between the at least one data extractor and the subject infrastructure, wherein:
   a) each of the at least one communication channel is dedicated to one of the at least one data extractor to ensure that failure of any one of the at least one communication channel affects only said associated one of the at least one data extractor;
   b) the at least one data extractor associated to the at least one communication channel is configured to create a connection with the subject infrastructure from the object infrastructure on which the at least one data extractor is installed.

In embodiments, the system with the main module of the at least one data extractor further comprises:
at least one configuration file, each of which stores a configuration for one of the plurality of child modules of the main module;
the configuration and update component of the parent process of the main module further configured to:
   a) upload said at least one configuration file to the storage of the subject infrastructure;
   b) detect changes in the at least one configuration object and update said at least one configuration file using the content of the at least one configuration object.

In embodiments, the system with the watchdog module of the at least one data extractor further comprises:
at least one configuration file, each of which stores a configuration for one of the plurality of child modules of the watchdog module;
the configuration and update component of the parent process of the watchdog module further configured to:
   a) upload said at least one configuration file to the storage of the subject infrastructure, wherein the resulting entity in said storage is referred to as “at least one configuration object”;
   b) detect changes in the at least one configuration object and update said at least one configuration file using the content of the at least one configuration object.

In embodiments, the system with the plurality of child modules of the main module of the data extractor further comprises:
an environment information module for extracting information about the at least one object infrastructure on which the at least one data extractor is installed.

In embodiments, the system with the plurality of child modules of the watchdog module of the data extractor further comprises:
an environment information module for extracting information about the object infrastructure on which the at least one data extractor is installed.

According to the present invention, there is also provided a computer-based software system for extracting or mutating data in at least one data source associated to at least one tenant, said at least one data source being located on an object infrastructure, the system comprising:
at least one data extractor connectable to the at least one data source for extracting the data from said data source or mutating said data in the said data source, said at least one data extractor being installed on the object infrastructure;
a subject infrastructure connectable to the at least one data extractor, wherein the at least one data extractor communicates (i) data extracted from the at least one data source and (ii) a log of operations of the at least one data extractor to the subject infrastructure;
   wherein the subject infrastructure comprises:
an identity and access management (IAM) module for (i) creating, mutating, or removing a plurality of IAM primitives and (ii) generating an event log of said creating, mutating, or removing of the plurality of IAM primitives for auditing, wherein said plurality of IAM primitives include:
   a) at least one subject infrastructure user;
   b) at least one role, and at least one access privilege to grant access and use of at least one infrastructure resource within the subject infrastructure to any associated at least one subject infrastructure user;
   c) at least one authentication credential associated to at least one subject infrastructure user;
wherein:
   d) each of the user in a subset of the at least one subject infrastructure user is associated to one of the at least one tenant and to one of the at least one data extractor and, and each of the user in said subset of user has (i) an associated at least one role, (ii) an associated at least one access privilege, or (iii) an associated at least one authentication credential;
   e) each of the user in (d) and the associated IAM primitives being together referred to as “at least one set of tenant data extraction IAM primitives”;
a tenant infrastructure management module (for provisioning or removing of:
   a) at least one set of tenant data extraction IAM primitives;
   b) at least one set of infrastructure resources, each set of infrastructure resources associated to the one of the at least one tenant which (a) is associated to, and access or usage of said infrastructure resources by the at least one data extractor which (a) is associated to being restricted by (a), said infrastructure resources comprising:
      i. a logically isolated storage;
      ii. at least one communication channel between the at least one data extractor and the subject infrastructure, each of said at least one communication channel being dedicated to one of the at least one data extractor associated to the one of the at least one tenant in (a);
the at least one set of tenant data extraction IAM primitives;
the at least one logically isolated storage storing objects comprising:
   a) the configuration files of the data extractor;
   b) the logs of operations of the main module and the watchdog module of the data extractor;
   c) the data extracted from the at least one data source;
the at least one communication channel;
an authentication credential lifecycle management module for coordinating the lifecycle of the at least one authentication credential of the at least one set of tenant data extraction IAM primitives;
an authentication credential activity logging module to log usage of the at least one authentication credential of the at least one set of tenant data extraction IAM primitives;
a configuration distribution module for exposing an initial configuration to the internet for consumption by one of the at least one data extractor during an installation of said data extractor on object infrastructure, wherein said initial configuration comprises the at least one authentication credential of the at least one set of tenant data extraction IAM primitives for the one of the at least one tenant associated to said data extractor being installed;
   wherein the at least one data extractor comprises:
the at least one authentication credential of the at least one set of tenant data extraction IAM primitives for the one of the at least one tenant associated to said data extractor, said at least one authentication credential granting access or usage to said data extractor to infrastructure resources of the subject infrastructure, said infrastructure resources comprising the at least one communication channel, and the at least one logically isolated storage.

Other objects, advantages and features of the present invention will become more apparent upon reading of the following non-restrictive description of specific embodiments thereof, given by way of example only with reference to the accompanying drawings.

### BRIEF DESCRIPTION OF THE DRAWINGS

FIG. 1 is a high-level deployment diagram providing an overview of the system disclosed, according to some embodiments.

FIG. 2, separated in FIG. 2A and FIG. 2B, is a low-level component diagram detailing the relationships between the various components composing a data extractor as well as some of the various components present in a subject infrastructure, according to some embodiments.

FIG. 3 is a component diagram detailing the various components of an Identity and Access Management (IAM) module, including a plurality of Identity and Access Management (IAM) primitives, according to some embodiments.

FIG. 4 highlights a group of Identity and Access Management (IAM) primitives of FIG. 3 to define the concept of “tenant data extraction IAM primitives”, according to some embodiments.

FIG. 5 is a sequence diagram illustrating a process of rotation of an authentication credential used by one of the at least one data extractor, and illustrating a sequence of steps performed by an authentication credentials lifecycle management module as part of a lifecycle of said authentication credential, according to some embodiments.

FIG. 6 is a sequence diagram illustrating a usage of an authentication credential by one of the at least one data extractor to access or use a resource (target, secondary target, nth target) located within a subject infrastructure as well as a logging of said access or use by an authentication credential activity logging module, according to some embodiments.

FIG. 7 is a sequence diagram illustrating an installation procedure of one of the at least one data extractor provided a prior existence of a tenant within a subject infrastructure, according to some embodiments.

### DETAILED DESCRIPTION OF EMBODIMENTS

The present invention is illustrated in further details by the following non-limiting examples.

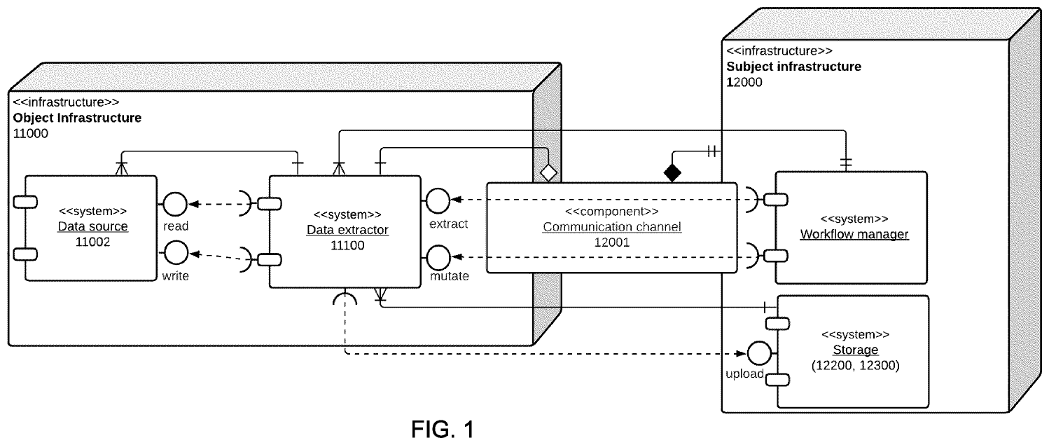

FIG. 1 is a high-level deployment diagram providing an overview of the system disclosed, according to some embodiments.

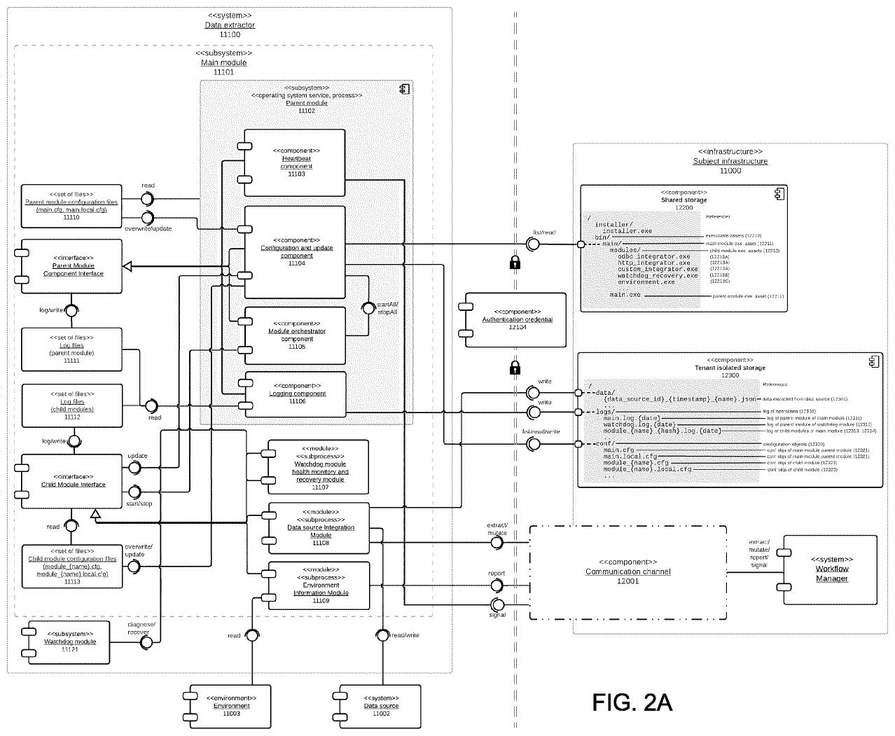

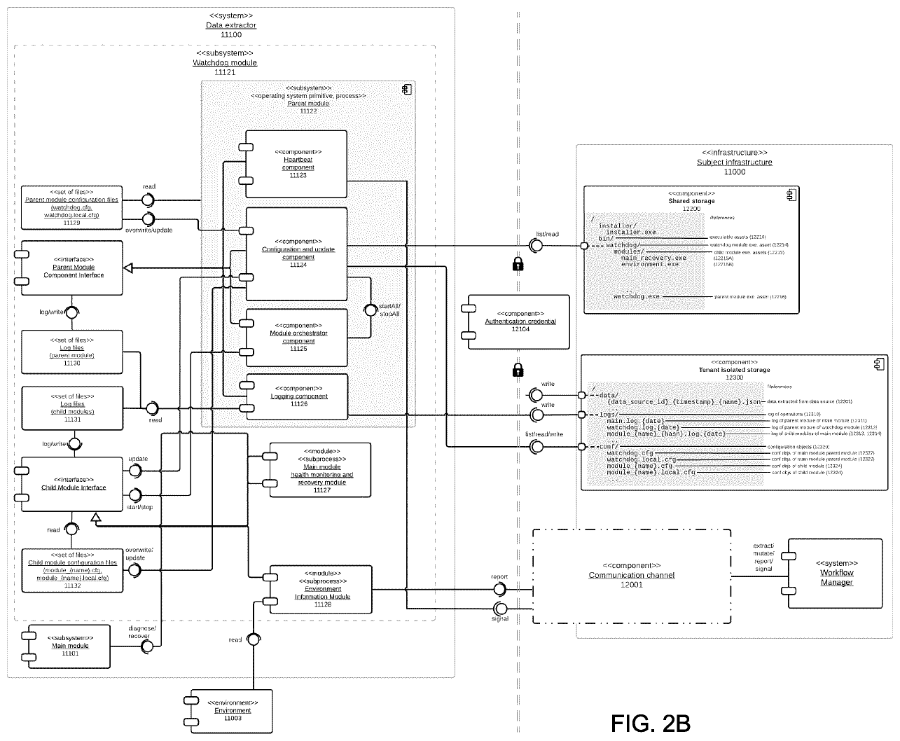

Referring to FIG. 2 in addition to FIG. 1, object infrastructure 11000 may be a private or closed system or may be set up by an entity such as a company or a public sector organization to provide one or more services to said entity or to a third-party. Said object infrastructure may host one or more physical or virtual computer machines, at least one of which having access to one or more data source 11002, hosted in an environment 11003.

Subject infrastructure 12000 may be a private or closed system or may be set up by an entity such as a company or a public sector organization to provide one or more service to a company, a public sector organization, or an embodiment of a legal person. In order to perform said service, the entity providing the service relying on the subject infrastructure 12000, or “integrator entity” may need to integrate data located within the one or more data source 11002 located on one or more object infrastructure 11000. Said integration of data may include extraction of data to the subject infrastructure 12000 or mutation of data within said data source 11003. In some embodiments, the one or more object infrastructure 11000 may belong to different legal entities or be in different private networks or closed systems.

In some embodiments, extraction or mutation of data in the one or more data source 11002 from outside the private or closed system of the one or more object infrastructure may be difficult or impossible due to network or infrastructure configuration. In such embodiments, an installation of a data extraction or mutation agent, “data extractor” 11100 may allow to perform extraction and mutation of data where it would have been otherwise impossible to do. In many embodiments, installation of the data extractor 11100 within the private or closed network on a long-lived machine may be sufficient to gain access to the data source and export the data to the subject infrastructure. In other embodiments, the data extractor 11100 may have to be installed on the same machine as the data source 11002, for example if the data source does not provide an interface that can be accessed over network.

Following the installation of a data extractor 11100, the data extractor may be configured to establish a connection over a secure communication channel 12001 to the subject infrastructure 12000, according to some embodiments. By having the data extractor establish the connection from within the object infrastructure 11000 to the subject infrastructure 12000, the embodiment can circumvent the network limitations preventing incoming connections on the object infrastructure, whereby allowing communication with a remote host located within the subject infrastructure. An embodiment of such communication channel should support full-duplex, real-time or near-real-time messages to allow a human operator of the subject infrastructure or an embodiment of the subject infrastructure or a subsystem thereof to communicate promptly with the data extractor in case of time sensitive operations, such as zero-day vulnerability patching, mirroring changes in data to the data source, etc. Optimal characteristics of an embodiment of such a communication channel would be to support long-lived, full-duplex, real-time or near-real-time connections to allow for two-way communications without the need to resort to asynchronous polling by the data extractor. Example technologies matching these requirements include the WebSocket protocol, gRPC protocol, and HTTP polling.

The data extractor 11100 may be configured to upload data extracted from a data source 11102 to an embodiment of storage 12300 for collection and use by the subject infrastructure 12000.

FIG. 2 is a low-level component diagram detailing the relationships between the various components composing a data extractor as well as some of the various components present in a subject infrastructure, according to some embodiments.

In some embodiments, extraction or mutation of data in the one or more data source 11002 over a long period of time (weeks, months or years) by a data extractor 11100 installed in the object infrastructure 11000 may be difficult to achieve due to concerns over reliability and resiliency of the data extractor. In effect, given that between various embodiments of the object infrastructure 11000 there may be significant differences in configurations of said infrastructure embodiments, the data extractor should maintain full functionality through changes in configurations of said infrastructures such as operating system upgrades, patch application, changes in available computational resources (CPU, memory, disk), etc. The data extractor should also reestablish full functionality following object infrastructure service interruptions such as shutdown of a machine on which the data extractor is installed.

As such the data extractor may implement techniques to ensure its reliability and resiliency.

For example, the data extractor 11100 may be executed as an operating system service 11102 to maximize its uptime. Operating the data extractor as an operating system service may allow the data extractor process 11102 to be (i) started alongside critical operating system processes on startup of the machine on which it is installed, (ii) executed with administrative privileges (iii) started independently of user logon, and (iv) automatically restarted by the operating system in case of failure, all three capacities being beneficial to reliability and resiliency.

Reliability of said operating system service process may be improved by delegating any non-reliability-essential, non-resiliency-essential, or prone to change functionality to child processes. Delegating said functionalities to child processes allow to minimize the code surface of the executable code of the operating system service process and as such reduce chances of failures in the parent process 11102. Said functionalities can then be packaged as separate executable assets which may follow a different release cycle than the parent process 11102 and thus be updated independently from the parent process.

If the delegation of said non-reliability-essential, non-resiliency-essential, or prone to change functionality to child processes is not implemented, it results that the parent process must be stopped, updated and restarted whenever changes in functionality are required. Such an update procedure comes with increased risks of failure, and thereby reduce the reliability of the data extractor 11100. In some embodiments, this need to change functionality may not arise often; nevertheless, the risk of mass failure of data extractors across a plurality of object infrastructures 11000, each possibly managed by different companies, public sector organizations, or legal person is a risk that may cost significant efforts and money to address would it occur, and as such the present design in this disclosure seeks to minimize the probability of this occurring.

Essential operational functionality of the parent process 11102 may include heartbeat exchange with the subject infrastructure 12000, and logging of operations to the subject infrastructure, according to some embodiments.

Heartbeat exchange may be considered as essential functionality since it provides the subject infrastructure 12000 or any natural person with sufficient access to said subject infrastructure 12000 the information of loss of heartbeat (failure of the parent process) or of an unhealthy heartbeat (failure in an essential operational functionality or child-process-delegated functionality), whereby said information may be used by said subject infrastructure 12000 or natural person to take action and resolve the failure.

Logging of operations may be considered as essential functionality since it provides the subject infrastructure 12000 or any natural person with sufficient access to said subject infrastructure 12000 detailed information about the operations of the data extractor for troubleshooting, data governance or audit purposes.

Delegation of non-reliability-essential, non-resiliency-essential, or prone to change functionality to child modules may need the parent process to extend said essential operational functionality with (i) orchestrating of said child processes, (ii) logging of operations of said child processes to the subject infrastructure 12000, as well as (iii) configuring and updating of executable assets and configuration files used by said child processes.

In some embodiments, essential operational functionality of the parent process 11102 may be organized as a set of components, said components being subsets of the code executed by the parent process. As such, the parent module may include (i) a heartbeat component 11103 to carry out heartbeat exchange with the subject infrastructure 12000, (ii) a logging component to handle logging of the parent process and of the child processes to the subject infrastructure 11106, a configuration and update component 11104 to handle configuring and updating of executable assets 12212 and configuration files of the parent module 11110 and executable assets 12213 and configuration files of the child modules 11113.

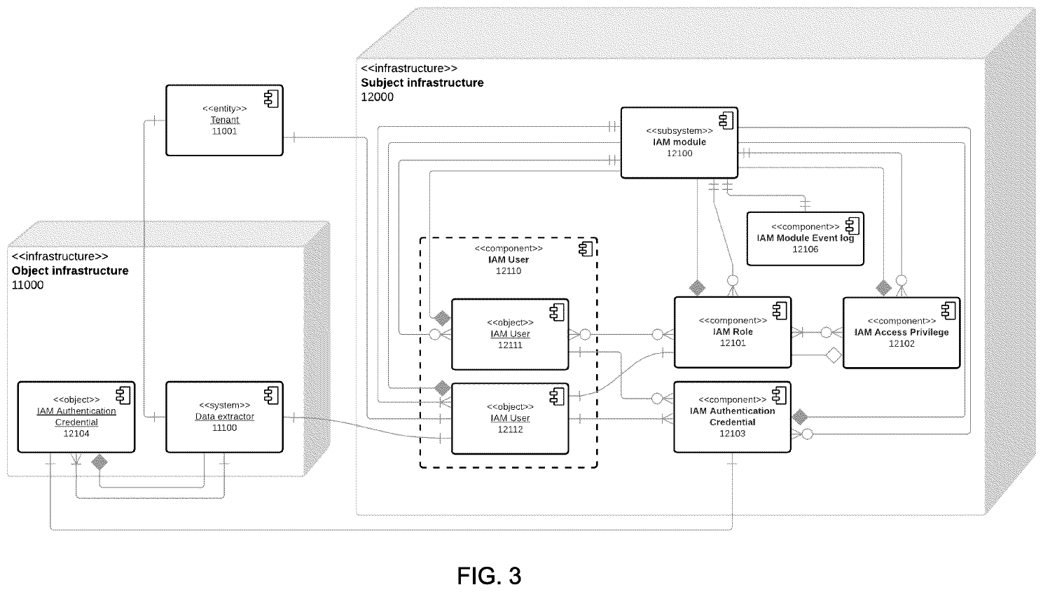

Referring now to FIG. 3, in addition to FIG. 2 and FIG. 1, in order to distribute the set of executable assets 12210 to the data extractors 11100, said set of executable assets may be stored in a single, shared storage 12200, or in a plurality of distinct storages 12300 in the subject infrastructure 12000, and be retrievable by one or more data extractor 11100. However, storing said set of executable assets in a centralized, shared storage 12200 may present benefits including simplifying deployment efforts, or minimizing configuration of the data extractors 11100 due to having a single source of truth for the executable assets. According to some embodiments, said executable assets may be versioned in said shared or distinct storage to allow the data extractor to downgrade at least one of the executable assets to a previous version if the configuration and update component 11104 fails to update the associated parent or child module to the latest version. In some embodiments, a semantic versioning or other versioning nomenclature, or a manifest object may be used to store compatibility between the different executable assets.

According to some other embodiments, said executable assets 12210 may also be stored outside of the subject infrastructure 12000.

As part of its child modules, the main module 11101 must include at least one data source integration module 11108 to perform its primary functionalities: data extraction or mutation from at least one data source 11002.

According to some embodiments, the main module may have more than one data source integration module 11108 in embodiments where the one or more data sources 11002 to integrate by the data extractor 11100 use different methods of connectivity or different drivers for exposing an extraction or mutation API. Example of methods of connectivity or drivers include ODBC connections, JDBC connections, REST APIs, GraphQL APIs, etc. A data source integration module 11108 may be configured to integrate more than one data sources 11002 in embodiments where said more than one data sources 11002 use the same method of connectivity or driver for exposing their extraction or mutation API.

According to some other embodiments, a data source integration module 11108 may integrate one or more data sources 11002 with different methods of connectivity or different drivers. However, separating integration of different methods of connectivity or different drivers across a plurality of data source integration modules 11108 may improve decoupling and compartmentalization, resulting in smaller, more reliable data source integration modules (11108).

In some embodiments, in order to improve resiliency of the data extractor 11100, each child module of the main module may have their respective configuration file 11113, each separate from the respective configuration files of other child modules 11113 and of the parent module of the main module 11110. This fragmentation in multiple configuration files rather than a single configuration file may improve resiliency by minimizing the probability that one or more module corrupts the otherwise single configuration file that all of the modules rely upon.

Resiliency of the data extractor 11100 may be improved by installing a watchdog alongside the operating system service parent module 11102 and its child modules, “main module” 11101, said watchdog being responsible for asserting the health of said main module, and (ii) attempting to repair said main module 11101 in case of failure. To maximize reliability of the watchdog, said watchdog may be installed as a process run by an operating system primitive, such as an operating system service or an operating system scheduled task.

Similarly to the operating system service, the reliability of watchdog process may be improved by delegating any non-reliability-essential, non-resiliency-essential, or prone to change functionality to child processes.

Essential operational functionality of the parent process of the watchdog 11121 may include heartbeat exchange with the subject infrastructure 12000, and logging of operations to the subject infrastructure, according to some embodiments.

Delegation of non-reliability-essential, non-resiliency-essential, or prone to change functionality to child modules may need the parent process of the watchdog 11121 to extend said essential operational functionality with (i) orchestrating of said child processes, (ii) logging of operations of said child processes to the subject infrastructure 12000, as well as (iii) configuring and updating of executable assets and configuration files used by said child processes.

In some embodiments, essential operational functionality of the parent process of the watchdog 11122 may be organized as a set of components, said components being subsets of the code executed by the parent process of the watchdog. As such, the parent module may include (i) a heartbeat component 11123, (ii) a logging component 11124, a configuration and update component 11125 to handle configuring and updating of executable assets 12212 and configuration files of the parent module 11129 and executable assets 12214 and configuration files of the child modules 11131.

As part of its child modules, the watchdog must include a main module health monitoring and recovery child module 11127 responsible for its primary functions: (i) performing a series of health check asserting the health of the main module 11101, and (ii) attempting recovery of detected failures in the main module 11101.

An embodiment of said main module 11101 health checks by the watchdog 11121 may include the following checks:
1. asserting that the main module is currently running as an operating system service process 11102, that said operating system service process is run with administrative privilege, or that said operating system service is properly configured to start automatically at boot;
2. asserting the executable assets 12211 and configuration files 12321, 12323 of each of the parent module and the child modules of the main module are present on the filesystem, exempt from corruption or tampering, or are up to date with the version published to shared storage 12200 in the subject infrastructure 12000;
3. asserting that the authentication credential 12104 used by the main module is well-formed and still before its expiration date;
4. asserting that each of the parent module and child modules of the main module 11101 are performing their functionality without failure or fault by reading their log or running each of said modules in diagnostic mode, said mode being packaged as part of each executable asset as a way to be executed to assert that the specific functionalities of the executable asset are performed without fault or failure.
   An embodiment of such recovery of detected failures in the main module 11101 may include:
1. restarting the main module's service process 11102 by using the appropriate operating system construct;
2. given a parent or child module of the main module failing to restart, collecting information about the failure and reporting said failure to the subject infrastructure 12000;
3. requesting or forcing the main module 11101 to update or overwrite its executable assets or its configuration files 11110, 11113 from the respective equivalent executable assets 12211 or configuration objects 12321, 12323 stored in the subject infrastructure 12000 storages 12200, 12300;
4. reinstalling the main module in a different directory;
5. rotating the authentication credential (12104) granted that said authentication credential has not expired yet;
6. executing failing modules in recovery mode, said mode being packaged as part of each executable asset as a way to be executed to recover from said-failing-modules-specific failures.

As part of its child modules, the watchdog module 11121 may include an environment information child module 11128 responsible for collecting information about the execution environment 11003, such as network conditions, CPU, memory or disk utilization, presence of known threats to the resiliency or reliability of the main module 11101 or watchdog module 11121, such as specific programs or procedures, etc.

In some embodiments, the watchdog module 11121 may report the result of its health checks on the main module 11101, the result of its attempts at recovery of failures in the main module 11101, or the information collected by its environment information child module 11128 to the subject infrastructure 12000.

As part of its child modules, the watchdog may include any number of additional child modules as deemed necessary for carrying out its functionalities or extending said functionalities with additional functionalities, according to some embodiments.

In some embodiments, in order to improve resiliency of the watchdog module 11121, each child module of the watchdog module may have their respective configuration file 11132, each separate from the respective configuration files of other child modules 11132 and of the parent module of the watchdog module 11129. This fragmentation in multiple configuration files rather than a single configuration file may improve resiliency by minimizing the probability that one or more module corrupts the otherwise single configuration file that all of the modules rely upon.

In order to improve the reliability and resiliency of the watchdog 11121, the main module 11101 may include a child module dedicated to (i) recurrently performing a series of health check asserting the health of the watchdog module 11121, and (ii) attempting recovery of detected failures in the watchdog module 11121.

An embodiment of said watchdog module 11121 health checks by the main module 11101 may include the following checks:
1. asserting that the watchdog module 11121 is currently running as an operating system service primitive 11122 according to the chosen embodiment, that said operating system primitive process is run with administrative privilege, or that said operating system primitive is properly configured;
2. asserting the executable assets 12214 and configuration files 12322, 12324 of each of the parent module and the child modules of the watchdog module are present on the filesystem, exempt from corruption or tampering, or are up to date with the version published to shared storage 12200 in the subject infrastructure 12000;
3. asserting that the authentication credential 12104 used by the watchdog module is well-formed and still before its expiration date;
4. asserting that each of the parent module and child modules of the watchdog module 11121 are performing their functionality without failure or fault by reading their log or running each of said modules in diagnostic mode, said mode being packaged as part of each executable asset as a way to be executed to assert that the specific functionalities of the executable asset are performed without fault or failure;
5. in some embodiments where the watchdog module 11121 is implemented as an operating system scheduled task, asserting that the last execution time of the watchdog module 11121 correlates with the configured execution schedule.
   An embodiment of such recovery of detected failures in the main module 11101 may include:
1. in some embodiments where the watchdog module 11121 is implemented as an operating system service, restarting the watchdog module's service by using the appropriate operating system construct;
2. given a parent or child module of the watchdog module failing to restart, collecting information about the failure and reporting said failure to the subject infrastructure 12000;
3. requesting or forcing the watchdog module 11121 to update or overwrite its executable assets or its configuration files 11129, 11132 from the respective equivalent executable assets 12214 or configuration objects 12322, 12324 stored in the subject infrastructure 12000 storages 12200, 12300;
4. reinstalling the watchdog module in a different directory;
5. rotating the authentication credential 12104 granted that said authentication credential has not expired yet;
6. executing failing modules in recovery mode, said mode being packaged as part of each executable asset as a way to be executed to recover from said-failing-modules-specific failures.

As part of its child modules, the main module 11101 may include an environment information child module 11109 responsible for collecting information about the execution environment 11003, such as network conditions, CPU, memory or disk utilization, presence of known threats to the resiliency or reliability of the main module 11101 or watchdog module 11121, such as specific programs or procedures, etc.

The presence of an environment information child module in both the watchdog module 11121 and the main module 11101 may allow both modules to act independently in their health monitoring, health checks, and failure recovery of the other module.

In some embodiments, the main module 11101 may report the result of its health checks on the watchdog module 11121 health, the result of its attempts at recovery of failures in the watchdog module 11121, or the information collected by its environment information child module 11109 to the subject infrastructure 12000.

Those skilled in the art will appreciate that the directory structure illustrated in the representation of the tenant isolated storage 12300 in FIG. 2 may differ from the embodiment presented in said figure, especially in embodiments where the tenant isolated storage 12300 serves multiple data sources 11002 or multiple data extractor 11100 concurrently.

FIG. 3 is a component diagram detailing the various components of an Identity and Access Management (IAM) module 12100, including a plurality of Identity and Access Management (IAM) primitives 12110, 12101, 12102, 12103, according to some embodiments.

In order to access the subject infrastructure 12000 and its infrastructure resources securely, each data extractor 11100 may authenticate itself against the subject infrastructure using an identity and access management (IAM) authentication credential 12104. In some embodiments, said authentication credential may enable the data extractor 11100 to assume the identity of an IAM user 12112 specifically associated to said data extractor. By assuming the identity of the IAM user 12112, the data extractor 11100 may gain access related to the IAM access privileges 12102 associated to one of the at least one IAM Roles 12101 associated with said IAM user 12112. Said IAM primitives 12110, 12112, 12101, 12102, 12103 may be “managed” (created, mutated, or removed) by the IAM module 12100.

In some embodiments, said IAM user 1211 associated to said data extractor may also be associated to a tenant 11001, thereby allowing the subject infrastructure 12000 to limit the access and usage of its infrastructure resources only to those provisioned for said tenant 11001.

In some embodiments of alternative structures to the invention disclosed, access to the subject infrastructure 12000 and its infrastructure resources may be shared across a plurality of data extractors 11100 irrespectively of which tenant 11001 is integrated by each of said plurality of data extractors 11100. In such an alternative structure, tenancy determination may be done after data is ingested by the subject infrastructure 12000.

These alternative structures may come with major security shortcomings however—in case of leak of the authentication credential 12104 of one of the plurality of data extractor sharing access to the subject infrastructure 12000, the entirety of shared infrastructure resources may be compromised, and in some embodiments of said alternative structures, may come with major data leaks if access privileges provided to data extractors are too expansive.

In order to mitigate such risk, the structure described in this disclosure ensures that each data extractor has its own set of authentication credential 12104, is only associated to one tenant 11001, and has only access to the infrastructure resources associated with said tenant or to infrastructure resources that can be shared safely without risking compromising tenant data security. Moreover, the structure described in this disclosure may be further hardened by implementing low-level access control lists wherever applicable. For example, the data extractor may have access to a tenant isolated storage 12200, but have only read access to certain objects stored within the storage, or only write access to certain other objects in said storage. Optimally, an embodiment should prioritize a structure of infrastructure resources, objects and more granular elements facilitating the minimization of minimum required access privileges for the data extractor to carry out its functions, within reason. For example, data extractors may only have read access to the configuration objects 12320 in storage, write access to data and log objects, and be denied all other operations against their associated tenant logically isolated storage 12300, including the overwriting of data or log objects where applicable.

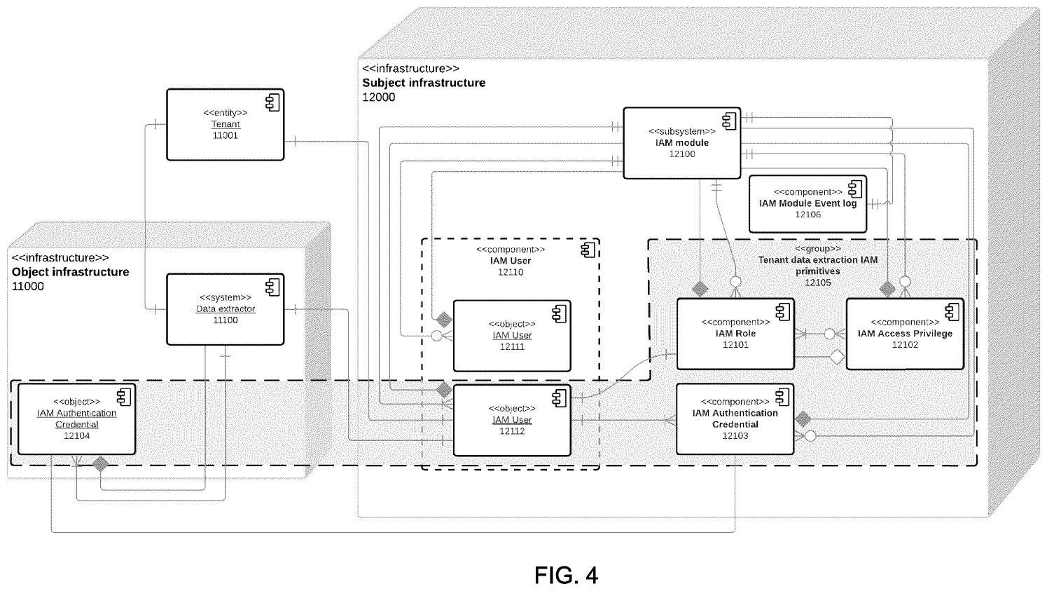

FIG. 4 highlights a group of Identity and Access Management (IAM) primitives of FIG. 3 to define the concept of “tenant data extraction IAM primitives” 12105, according to some embodiments.

According to some embodiments, the concept of “tenant data extraction IAM primitives” 12105 include IAM primitives associated to a tenant 11001 and a data extractor 11100, said primitives including an IAM User 12112, at least one IAM Role 12101, at least one IAM Access Privilege 12102, and at least one IAM Authentication Credential 12103, downloaded to the object infrastructure 11000 as an object 12104 such as a file, registry key or other form of storage on the object infrastructure 11000.

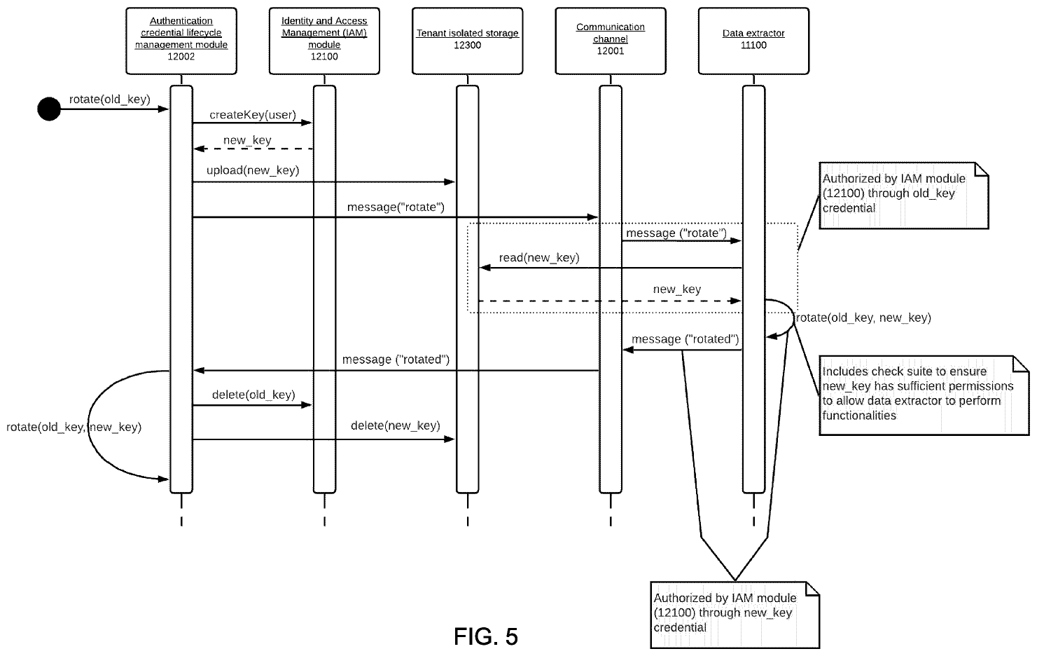

FIG. 5 is a sequence diagram illustrating a process of rotation of an authentication credential 12104 used by one of the at least one data extractor 11100, and illustrating a sequence of steps performed by an authentication credentials lifecycle management module 12002 as part of a lifecycle of said authentication credential 12104, according to some embodiments.

In order to further reduce the size of a security breach in the event of an authentication credential 12104 leak, an embodiment may opt to rotate said authentication credential 12104 frequently. For example, an embodiment may opt to rotate credentials every hour, few hours or every day. Such a measure may reduce the size of the breach by shortening the time window during which the attacker has access to a valid authentication credential 12104.

As such, in some embodiments, the subject infrastructure 12000 may have an automated rotation system designed in such a way such as to invalidate existing valid authentication credentials 12104 and provide data extractors 11100 with a new, valid authentication credential instead.

The sequence described in FIG. 5 represent the application of such an automated rotation system, according to some embodiments. Given a trigger event, the authentication credential lifecycle management module 12002 may generate a new authentication credential 12103 and upload the resulting authentication credential 12104 to a storage only accessible by said data extractor 11100, such as the tenant logically isolated storage 12300. Following upload, the authentication credential lifecycle management module 12002 may inform the data extractor 11100 that a new authentication credential 12104 is available and that the currently used authentication credential 12104 has been flagged for rotation and invalidation. Given this information, the data extractor 11100 may download the new authentication credential 12104 from the secure storage 12300, perform a check suite to ensure that said new authentication credential 12104 is indeed viable to authorize the data extractor 11100 to perform its operations with the subject infrastructure 12000, and notify the authentication credential lifecycle management module 12002 back that rotation has been done. The authentication credential lifecycle management module 12002 may then delete the new authentication credential from the secure storage and delete or invalidate the old key from the IAM module 12100, thereby finishing the authentication credential rotation sequence.

An embodiment implementing the above sequence ensures that only an entity with a valid authentication credential 12104 can gain access to a new authentication credential 12104. An embodiment of the above sequence may further ensure the security of the operation by halting the rotation sequence if the authentication credential 12104 bearer does not respond adequately or within a reasonable time frame and may flag the authentication credential 12104 as compromised and trigger an alert for a human person to take appropriate action.

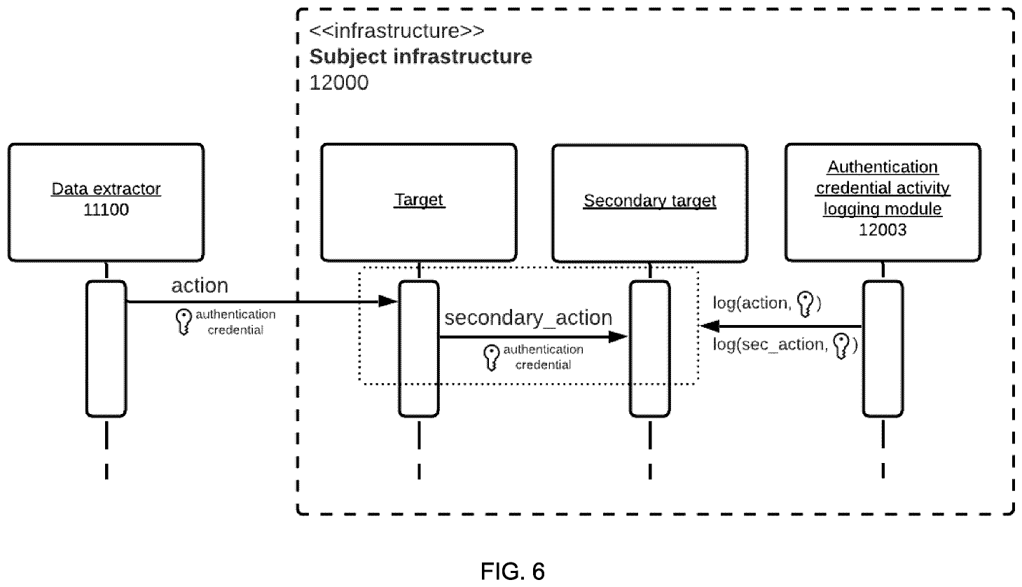

FIG. 6 is a sequence diagram illustrating a usage of an authentication credential 12104 by one of the at least one data extractor 11100 to access or use a resource (target, secondary target, nth target) located within a subject infrastructure 12000 as well as a logging of said access or use by an authentication credential activity logging module 12003 for purposes including audit or troubleshooting, according to some embodiments.

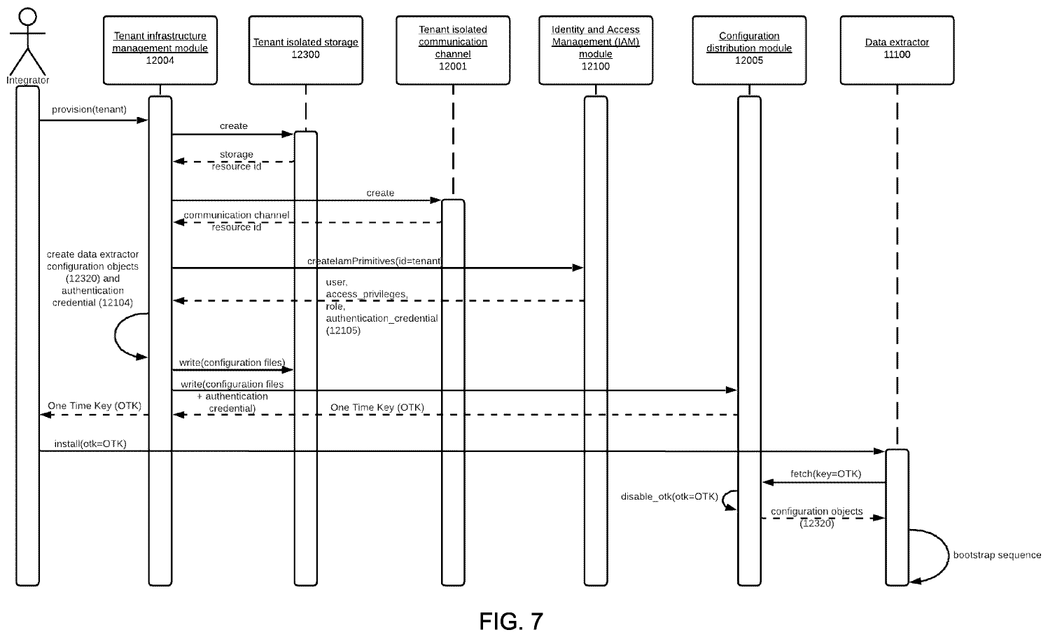

FIG. 7 is a sequence diagram illustrating an installation procedure of one of the at least one data extractor 11100 provided a prior existence of a tenant within a subject infrastructure (12000), according to some embodiments.

The sequence of steps presented in FIG. 7 demonstrates how an embodiment may automatically provision relevant tenant infrastructure resources 12300, 12001, 12105 in the subject infrastructure 12000 such that the embodiment may benefit from the benefits discussed in above paragraphs [00112], [00113] and [00116]

Furthermore, the sequence of steps presented in FIG. 7 removes the need for the integrator to access the initial authentication credential 12104 and configuration objects 12320, “initial configuration objects”, to configure the data extractor 11100. Indeed, the sequence of steps in FIG. 7 automatically generates a single-use, unique key, “one time key (OTK)” acting as a resource location identifier for said initial configuration objects that is then provided to the integrator to input in the install wizard of the data extractor 11100 for said install wizard to automatically download said initial configuration objects. The one time key may be provided to the integrator over a secure channel. For optimal security, the one time key should be a long, high entropy string of characters, such as a GUID. A high entropy key, combined with a single-usage model and with a limited expiry window provides reasonably strong security given the low collision rate of said high entropy key and the limited time to attempt brute force discovery of valid keys. An embodiment may even employ rate limiting or firewall security to completely deny brute force attacks from external hosts against the configuration distribution module 12005.

The sequence of steps presented in FIG. 7 further improves security of initial configuration objects as it removes the need for the integrator to enter some form of reusable authentication credentials on the object infrastructure 11000. This is important because in cases where the machine on which the data extractor 11100 is installed is compromised, the entry of some form of reusable authentication credentials authenticating the bearer as the integrator may open an attack vector against the subject infrastructure 12000 and its content if the compromised machine has a keylogger or some sort of clipboard reader software installed. Granted, such an attack vector may be mitigated by multi factor authentication, but this still leaves the door open to other attack vectors such as social engineering, credential mismanagement, etc.

While it may not provide additional significant benefits, given the high level of security of the sequence of steps presented in FIG. 7, some embodiments may use a modified version of this sequence to perform authentication credential 12104 rotation. For example, instead of uploading the new authentication credential 12104 to the tenant isolated storage 12300, said embodiments may write the new authentication credential 12104 to the configuration distribution module 12005 and then send the associated one time key to the data extractor 11100 over its communication channel 12001. The data extractor 11100 may then obtain the authentication credential 12104 from the configuration distribution module 12005 by providing the previously obtained one time key.

An embodiment of the system disclosed herein may additionally implement data governance, auditing, and anomaly detection mechanisms using one or more of the logs described by the invention, including the logs of operations of the data extractor 12310, the event log of the IAM module 12106, and the log produced by the authentication credential activity logging module 12003. In some embodiments, elements of the subject infrastructure 12000 discussed in this disclosure may also produce logs of their own that may be relevant to some of said additional mechanisms.

Those skilled in the art will appreciate that the system described herein is merely illustrative and is not intended to limit the scope of the techniques as described herein.

Various modifications and changes may be made as would be obvious to a person skilled in the art having the benefit of this disclosure. It is intended that the invention embrace all such modifications and changes and, accordingly, the above description to be regarded in an illustrative rather than a restrictive sense.

1. A method for improving reliability and resiliency of a computer-based software system installed on an object infrastructure (11000) managed by an external party, the method comprising:
a. packaging said computer-based software system as a first executable asset (12213A);
b. installing an executable asset (12212) of a first parent module (11102) as an operating system service on the object infrastructure (11000), said first parent module (11102) being configured to operate at least one child module (11107, 11108, 11109), each of said at least one child module comprising a child process executing an executable asset selected from a first set of executable assets (12213A, 12213B, 12213C);
c. installing an executable asset (12216) of a second parent module (11122) as an operating system primitive on the object infrastructure (11000), said second parent module (11122) being configured to operate at least one child module (11127, 11128), each of said child module comprising a child process executing an executable asset from a second set of executable assets (12215A, 12215B);
d. configuring the first parent module (11102) to include a first child module (11108) executing the executable asset (12213A) of the computer-based software system, and a second child module (11107) executing a second executable asset (12213B) being configured to monitor health of the second parent module (11122) and said at least one child module (11127, 11128) of the second parent module (11122) and attempt recovery of a detected failure in the second parent module (11122) and in said at least one child module (11127, 11128) of the second parent module (11122);
e. configuring the second parent module (11122) to include one child module executing a third executable asset (12215A) being configured to monitor health of the first parent module (11102) and at least one child module (11107, 11108, 11109), and attempt recovery of the detected failure;
f. monitoring the health of the first parent module (11102), the second parent module (11122), and their respective at least one child module (11107, 11108, 11109, 11127, 11128); and
g. attempting recovery of the detected failure in the first parent module (11102), the second parent module (11122), and their respective at least one child module (11107, 11108, 11109, 11127, 11128).

2. The method of claim 1, further comprising:
h. configuring the first parent module (11102) to automatically update the first set of executable assets (12213A, 12213B, 12213C) of its at least one child module (11107, 11108, 11109) whenever the first set of executable assets (12213A, 12213B, 12213C) for said at least one child module (11107, 11108, 11109) are published to a configured location (12200), said update of step h. comprising executing a check suite to assert the first set of executable assets (12213A, 12213B, 12213C) do not introduce a failure;
i. configuring the second parent module (11122) to automatically update the second set of executable assets (12215A, 12215B) of its at least one child module (11127, 11128) whenever the second set of executable assets (12215A, 12215B) for said at least one child module (11127, 11128) is are published to the configured location (12200), said update of step i. comprising executing a check suite to assert the second set of executable assets (12215A, 12215B) do not introduce a failure;
j. automatically updating the first set of executable assets (12213A, 12213B, 12213C) of the at least one child module (11107, 11108, 11109) of the first parent module (11102) whenever the first set of executable assets (12213A, 12213B, 12213C) for said at least one child module (11107, 11108, 11109) is published to the configured location (12200), said updating of step i. comprising executing a check suite to assert the first set of executable assets (12213A, 12213B, 12213C) do not introduce one or more failures; and
k. automatically updating the second set of executable assets (12215A, 12215B) of the at least one child module (11127, 11128) of the second parent module (11122) whenever a new executable asset (12215) for said at least one child module (11127, 11128) is published to the configured location (12200), said updating of step k. comprising executing a check suite to assert the second set of executable assets (12215A, 12215B) do not introduce one or more failures.

3. The method of claim 1, further comprising:
h. notifying a subject infrastructure (12000) of a detected failure; and
i. notifying the subject infrastructure (12000) of a result of the attempted recovery in step (g).

4. A method for securely extracting or mutating data associated to a tenant (11001) in at least one data source (11002) located in an object infrastructure (11000), from a subject infrastructure (12000), the method comprising:
a. configuring the subject infrastructure (12000) provision logically isolated infrastructure resources (12001, 12105, 12300) dedicated to the tenant (11001), said resources comprising a communication channel (12001), a set of tenant data extraction Identity and Access Management (IAM) primitives (12105), and a tenant logically isolated storage (12300);
b. granting external access to said infrastructure resources (12001, 12105, 12300) to entities authenticating as a user (12112) comprised in said set of tenant data extraction IAM primitives (12105);
c. writing an authentication credential (12104) for said user (12112) to a configuration distribution module (12005), said configuration distribution module returning a single-use, high-entropy, unique key, “one time key”;
d. installing a computer-based software system, “data extractor” (11100) on the object infrastructure to perform extraction or mutation of said data associated to the tenant (11001) in the at least one data source (11002);
e. configuring said data extractor (11100) to connect to said at least one data source (11002);
f. configuring said data extractor (11100) to retrieve said authentication credential (12104) from said configuration distribution module (12005), thereby using the single-use one time key, and providing said computer-based software system (11100) with access to the logically isolated infrastructure resources (12001, 12105, 12300);
g. using said communication channel (12001) to communicate between the data extractor (11100) and the subject infrastructure (12000) to (i) receive extraction or mutation commands, (ii) execute said extraction or mutation commands, and (iii) respond where applicable; and
h. using said tenant logically isolated storage (12300) to upload extracted data to the subject infrastructure (12000).

5. The method of claim 4, further comprising a recurrent and automatic rotation of said authentication credential (12104), said rotation comprising:
i. distributing a new authentication credential (12104) to the tenant logically isolated storage (12300);
j. communicating to the data extractor (11100) that a new authentication credential (12104) is available in the tenant logically isolated storage (12300);
k. the data extractor (11100) downloading the new authentication credential (12104) to the object infrastructure (11000);
l. the data extractor (11100) performing a check suite to assert that the new authentication credential (12104) has sufficient access privilege on the subject infrastructure (12000) to allow the data extractor (11100) to perform all of at least one function of said data extractor (11100);
m. the data extractor (11100) communicating to the subject infrastructure (12000) that it has rotated its authentication credential (12104) with the new authentication credential (12104); and
n. the subject infrastructure (12000) expiring the authentication credential (12104) rotated out by the data extractor (11100).

6. A computer-based software system for extracting or mutating data in at least one data source (11002) associated to at least one tenant (11001), said at least one data source being located on an object infrastructure (11000), the system comprising:
a. at least one data extractor (11100) connectable to the at least one data source (11002) for extracting the data from said data source or mutating said data in the said data source, said at least one data extractor being installed on the object infrastructure;
b. a subject infrastructure (12000) connectable to the at least one data extractor, wherein the at least one data extractor (11100) communicates (i) data extracted from the at least one data source (11002) and (ii) a log of operations (12310) of the at least one data extractor (11100) to the subject infrastructure;
wherein the at least one data extractor comprises:
a main module (11101) for performing the extraction or mutation of the data in the at least one data source (11002), said main module comprising:
a) a parent module (11102) executed as an operating system service process from a first corresponding executable asset (12212);
b) a configuration file (11110) to store a configuration of the parent module (11102);
c) a plurality of child modules (11107, 11108), each of which being separated from the parent module of the main module and from each other by each being executed from a corresponding executable asset of a first set of executable assets (12213A, 12213B, 12213C) as a child process of the process of the parent module of the main module;
a watchdog module (11121) for monitoring health of the main module (11101) and attempting recovery of detected failures in said main module, said watchdog module comprising:
a) a parent module (11122) executed as an operating system primitive including an operating system service process or an operating system scheduled task process from a second corresponding executable asset (12214);
b) a configuration file (11124) to store a configuration of the parent module (11122);
c) a plurality of child modules (11127), each of which being separated from the parent module of the watchdog module and from each other by each being executed from a corresponding executable asset of a second set of executable assets (12215A, 12215B) as a child process of the process of the parent module of the watchdog module (11122);
the parent process of the main module (11102), comprising:
a) a heartbeat component (11103) for sending a heartbeat signal to the subject infrastructure (12000) to inform said subject infrastructure that the parent process of the main module of the at least one data extractor has liveness;
b) a configuration and update component (11104) for
i. updating the configuration file (11110) of the parent module of the main module (12321), and a configuration file (11113) of the executable assets of the plurality of child modules of the main module (12213);
ii. uploading the configuration file (11110) of the parent module of the main module (11102) to the subject infrastructure (12000);
c) a module orchestrator component (11105) for bootstrapping, starting, stopping and restarting the child processes of the plurality of child modules of the main module;
d) a logging component (11106) for uploading logs of the parent module of the main module (12311), and logs of the plurality of child modules of the parent module of the main module (12313) to the subject infrastructure (12000);
the plurality of child modules of the main module, comprising:
a) a watchdog module health monitoring and recovery module (11107) for recurrently performing a series of health checks on the watchdog module (11121), and attempting recovery of detected failures in the watchdog module (11121);
b) at least one data source integration module (11108) for extracting the data from the at least one data source (11002) or mutating the data within said data source (11002);
the parent process of the watchdog module (11122) comprising:
a) a heartbeat component (11123) for sending a heartbeat signal to the subject infrastructure (12000) to inform said subject infrastructure that the parent process of the watchdog module of the at least one data extractor has liveness;
b) a configuration and update component (11124) for:
i. updating the configuration file (11132) of the parent module of the watchdog module (12322), the executable assets of the plurality of child modules of the watchdog module (12215);
ii. uploading the configuration file of the parent module of the watchdog module (12322) to the subject infrastructure (12000);
c) a module orchestrator component (11125) for bootstrapping, starting, stopping and restarting the child processes of the plurality of child modules of the watchdog module;
d) a logging component (11126) for uploading logs produced by the parent module of the watchdog module (12312), logs of the plurality of child modules of the parent module of the watchdog module (12314) to the subject infrastructure (12000);
the plurality of child modules of the watchdog module, comprising:
a) a main module health monitoring and recovery module (11127) for recurrently performing a series of health checks on the main module (11101), and attempting recovery of detected failures in the main module (11101);
wherein the subject infrastructure (12000) comprises:
a storage (12200, 12300) comprising:
a) the executable assets used by the at least one data extractor (12210), said executable assets comprising the executable assets for the main module, watchdog module, and their respective components and child modules;
b) a set of objects for each one of the at least one data extractor (12310), each set of objects comprising:
i. the configuration files of the at least one data extractor (12320);
at least one communication channel (12001) between the at least one data extractor and the subject infrastructure (12000), wherein:
a) each of the at least one communication channel is dedicated to one of the at least one data extractor (11100) to ensure that failure of any one of the at least one communication channel affects only said associated one of the at least one data extractor (11100);
b) the at least one data extractor associated to the at least one communication channel is configured to create a connection with the subject infrastructure (12000) from the object infrastructure (11000) on which the at least one data extractor (11100) is installed.

7. The system of claim 6, wherein the main module (11101) of the at least one data extractor (11100) further comprises:
at least one configuration file (12323), each of which stores a configuration for one of the plurality of child modules of the main module (11107, 11108, 11109);
the configuration and update component (11104) of the parent process of the main module (11101) further configured to:
a) upload said at least one configuration file (12323) to the storage (12300) of the subject infrastructure (12000);
b) detect changes in the at least one configuration file (12323) and update said at least one configuration file (12323) using the content of the at least one configuration file (12323).

8. The system of claim 6, the watchdog module (11121) of the at least one data extractor (11100) further comprises:
at least one configuration file (12324), each of which stores a configuration for one of the plurality of child modules of the watchdog module (11121);
the configuration and update component (11124) of the parent process of the watchdog module (11122) further configured to:
a) upload said at least one configuration file (12324) to the storage (12300) of the subject infrastructure (12000), wherein the resulting entity in said storage is referred to as “at least one configuration object” (12324);
b) detect changes in the at least one configuration object (12324) and update said at least one configuration file (12324) using the content of the at least one configuration object (12324).

9. The system of claim 6, wherein the plurality of child modules of the main module of the data extractor (11101) further comprises:
an environment information module (11109) for extracting information about the object infrastructure (11000) on which the at least one data extractor is installed.

10. The system of claim 6, wherein the plurality of child modules of the watchdog module of the data extractor (11121) further comprises:
an environment information module (11128) for extracting information about the object infrastructure (11000) on which the at least one data extractor is installed (11100).

11. A computer-based software system for extracting or mutating data in at least one data source (11002) associated to at least one tenant (11001), said at least one data source being located on an object infrastructure (11000), the system comprising:
at least one data extractor (11100) connectable to the at least one data source (11002) for extracting the data from said data source or mutating said data in the said data source (11002), said at least one data extractor being installed on the object infrastructure (11000);
a subject infrastructure (12000) connectable to the at least one data extractor, wherein the at least one data extractor (11100) communicates (i) data extracted from the at least one data source and (ii) a log of operations of the at least one data extractor to the subject infrastructure;
wherein the subject infrastructure (12000) comprises:
an identity and access management (IAM) module (12000) for (i) creating, mutating, or removing a plurality of IAM primitives (12101, 12102, 12103, 12110) and (ii) generating an event log (12106) of said creating, mutating, or removing of the plurality of IAM primitives (12101, 12102, 12103, 12110) for auditing, wherein said plurality of IAM primitives include:
a) at least one subject infrastructure user (12110);
b) at least one role (12101), and at least one access privilege (12102) to grant access and use of at least one infrastructure resource within the subject infrastructure (12000) to any associated at least one subject infrastructure user (12000);
c) at least one authentication credential (12103) associated to at least one subject infrastructure user (12110);
wherein:
d) each user in a subset (12112) of the at least one subject infrastructure user (12110) is associated to one of the at least one tenant (11001) and to one of the at least one data extractor (11100), and each user in said subset of users (12112) has (i) an associated at least one role (12101), (ii) an associated at least one access privilege (12102), or (iii) an associated at least one authentication credential (12104);
e) each user in (d) (12112) and the associated IAM primitives (12101, 12102, 12104) being together referred to as “at least one set of tenant data extraction IAM primitives” (12105);
a tenant infrastructure management module (12004) for provisioning or removing of:
a) at least one set of tenant data extraction IAM primitives (12105);
b) at least one set of infrastructure resources, each set of infrastructure resources associated with one of the at least one tenant (11001) and at least one of said at least one set of tenant data extraction IAM primitives (12105), access or usage of said infrastructure resources by the at least one data extractor (11100) being granted by said at least one set of tenant data extraction IAM primitives (12105), said infrastructure resources comprising:
i. a logically isolated storage (12300);
ii. at least one communication channel (12001) between the at least one data extractor and the subject infrastructure (12000), each of said at least one communication channel (12001) being dedicated to one of the at least one data extractor (11100) associated to the one of the at least one tenant (11001) in (a);
the at least one set of tenant data extraction IAM primitives (12105);
the logically isolated storage (12300) storing objects comprising:
a) configuration files of the data extractor (12320);
b) logs of operations of a main module and a watchdog module of the data extractor (12310);
c) the data extracted from the at least one data source (12301);
the at least one communication channel (12001);
an authentication credential lifecycle management module (12002) for coordinating a lifecycle of the at least one authentication credential (12104) of the at least one set of tenant data extraction IAM primitives (12105);
an authentication credential activity logging module (12003) to log usage of the at least one authentication credential (12104) of the at least one set of tenant data extraction IAM primitives (12105);
a configuration distribution module (12005) for exposing an initial configuration to the internet for consumption by one of the at least one data extractor (11100) during an installation of said data extractor (11100) on the object infrastructure (11000), wherein said initial configuration comprises the at least one authentication credential (12104) of the at least one set of tenant data extraction IAM primitives (12105) for the one of the at least one tenant (11001) associated to said data extractor (11100) being installed;
wherein the at least one data extractor comprises:
the at least one authentication credential (12104) of the at least one set of tenant data extraction IAM primitives (12105) for the one of the at least one tenant (11001) associated to said data extractor (11100), said at least one authentication credential (12104) granting access or usage to said data extractor (11100) to infrastructure resources of the subject infrastructure (12000), said infrastructure resources comprising the at least one communication channel (12001), and the at least one logically isolated storage (12300).
